(19) United States
(12) Patent Application Publication
(10) Pub. No.: US 2016/0296681 A1
(43) Pub. Date: Oct. 13, 2016

(54) FLUID MEASUREMENT ACCESSORY FOR BREAST PUMPS

(71) Applicant: Naya Health, Inc., Redwood City, CA (US)

(72) Inventors: Nathaniel Gaskin, Palo Alto, CA (US); Jeffery B. Alvarez, Redwood City, CA (US); Alex Goldenberg, San Francisco, CA (US); Greg Stahler, Belmont, CA (US)

(21) Appl. No.: 15/094,690

(22) Filed: Apr. 8, 2016

Related U.S. Application Data
(60) Provisional application No. 62/144,854, filed on Apr. 8, 2015.

(51) Int. Cl. A61M 1/06

(52) U.S. Cl. CPC A61M 1/062; A61M 2205/702; A61M 2205/3569; A61M 2205/52; A61M 2205/3327

(57) ABSTRACT

Methods and apparatus are provided for measuring one or more aspects of breast milk expression. A sensing adaptor comprises a housing having a first end, a second end, and a central channel defined therethrough. The first coupling mechanism is disposed adjacent the first end, and configured to removably couple to a pumping device. The second coupling mechanism is disposed adjacent the second end, and configured to removably couple to a collection vessel receiving the expressed breast milk from the pumping device. The sensing adaptor further comprises a sensor coupled to the housing, the sensor configured to measure one or more aspects of the expressed breast milk as the expressed breast milk enters the collection vessel through the central channel from the pumping device.

### CROSS-REFERENCE

The present application is a non-provisional of, and claims the benefit of U.S. Provisional Patent Application No. 62/144,854, filed on Apr. 8, 2015 [attorney docket no. 44936-710.101], the entire content of which is incorporated herein by reference.

This application is related to the following co-pending provisional and non-provisional patent applications: U.S. patent application Ser. No. 14/221,113, filed on Mar. 20, 2014 [attorney docket no. 44936-703.201], U.S. patent application Ser. No. 14/616,557, filed on Feb. 6, 2015 [attorney docket no. 44936-704.201], U.S. patent application Ser. No. 14/793,606, filed on Jul. 7, 2015 [attorney docket no. 44936-705.201], U.S. patent application Ser. No. 14/793,613, filed on Jul. 7, 2015 [attorney docket no. 44936-706.201], U.S. patent application Ser. No. 14/793,617, filed on Jul. 7, 2015 [attorney docket no. 44936-707.201], U.S. patent application Ser. No. 14/858,924, filed on Sep. 18, 2015 [attorney docket no. 44936-709.201], and U.S. Provisional Patent Application No. 62/144,854, filed on Apr. 8, 2015, the entire contents of which are incorporated herein by reference.

### BACKGROUND OF THE INVENTION

1. Field of the Invention

The present invention generally relates to medical devices and methods, and more particularly relates to devices and methods for expression and collection of human breast milk.

Breast pumps are commonly used to collect breast milk in order to allow mothers to continue breastfeeding while apart from their children. In order to understand their milk production and ensure that the production is maintained at a sufficient level, mothers often keep records of their pumping sessions manually, for example in journals or spreadsheets. Manual keeping of records can be cumbersome and prone to inaccuracies or lapses in record-keeping.

It would be desirable to provide a way for mothers to automatically keep track of their milk production and of the expressed milk inventory. It would be further desirable for the means to quantify breast milk production to be adaptable for use with various types of breast pumps. Automatic milk production quantification and inventory tracking via communication with mobile devices are further desirable for enhanced user convenience.

At least some of these objectives will be satisfied by the devices and methods disclosed below.

2. Description of the Background Art

The following US patents are related to expression and collection of human breast milk: U.S. Pat. Nos.: 6,673,036; 6,749,582; 6,840,918; 6,887,210; 7,875,000; 8,118,772; and 8,216,179.

### SUMMARY OF THE INVENTION

The present invention generally relates to medical devices and methods, and more particularly relates to devices and methods for expression and collection of human breast milk.

Methods and apparatus are provided for measuring one or more aspects of breast milk expression. A sensing adaptor comprises a housing having a first end, a second end, and a central channel defined therethrough. The first coupling mechanism is disposed adjacent the first end, and configured to removably couple to a pumping device. The second coupling mechanism is disposed adjacent the second end, and configured to removably couple to a collection vessel receiving the expressed breast milk from the pumping device. The sensing adaptor further comprises a sensor coupled to the housing, the sensor configured to measure one or more aspects of the expressed breast milk as the expressed breast milk enters the collection vessel through the central channel from the pumping device.

In one aspect, an apparatus for measuring expressed breast milk is provided. The apparatus comprises a housing having a first end, a second end, and a central channel defined therethrough. The apparatus further comprises a first coupling mechanism disposed adjacent the first end, the first coupling mechanism configured to removably couple to a pumping device, and a second coupling mechanism disposed adjacent the second end, the second coupling mechanism configured to removably couple to a collection vessel receiving the expressed breast milk from the pumping device. The apparatus further comprises a sensor coupled to the housing, wherein the sensor is configured to measure one or more aspects of the expressed breast milk as the expressed breast milk passes from the pumping device through the central channel into the collection vessel.

In some embodiments, the apparatus further comprises a processing unit in communication with the sensor. The processing unit may comprise a communication module configured to communicate with one or more of a computing device or a server. The communication module may be configured to transmit the measurement data generated by the sensor to one or more of a computing device or a server. The processing unit may be configured to analyze measurement data generated by the sensor, and the communication module is configured to transmit the analyzed measurement data to one or more of a computing device or a server. In some embodiments, the apparatus further comprises a power source operatively coupled to the sensor. The housing may comprise a shroud surrounding one or more of the sensor, a processing unit, and a power source, thereby protecting one or more of the sensor, processing unit, and power source from external forces.

The housing may comprise an exterior portion and an interior portion, the exterior portion comprising the first coupling mechanism and the interior portion comprising the sensor and the second coupling mechanism. The exterior portion and the interior portion may be coupled together, for example such that the exterior portion and the interior portion are fixed in position relative to one another during operation of the apparatus.

The first coupling mechanism and the second coupling mechanism may comprise coupling mechanisms selected from screw threads, quarter turn couplings, bayonet couplings, or interference fits. For example, the first coupling mechanism may comprise male screw threads, and the second coupling mechanism comprises female screw threads.

In some embodiments, the sensor comprises a strain gauge. In some embodiments, the strain gauge may be disposed on a lateral member of the housing, wherein the lateral member may be coupled to the second coupling mechanism. The strain gauge may be configured to measure strain placed on the second coupling mechanism by the collection vessel coupled thereto, thereby generating measurement data indicative of a volume of expressed milk. The housing can be configured to isolate a load exerted by the collection vessel. For example, the housing may comprise a lip coupled to the lateral member, the lip configured to engage a top surface of the collection vessel coupled to the second coupling mechanism, such that the top surface of the collection vessel is flush against a bottom surface of the lip. The lateral member may be configured annularly around the central channel, or may comprise one or more elongated beams. The strain gauge may comprise a plurality of strain gauges disposed on a plurality of locations on the lateral member.

In some embodiments, the strain gauge may be disposed on a valve, wherein the valve may be coupled to the housing such that the valve permits passage of the expressed breast milk through the central channel. The strain gauge may be configured to measure strain placed on the valve by the expressed breast milk, thereby generating measurement data indicative of a volume of expressed milk.

In some embodiments, the sensor comprises an accelerometer. The accelerometer may comprise a valve accelerometer disposed on a valve, wherein the valve may be coupled to the housing such that the valve permits passage of the expressed breast milk through the central channel. The valve accelerometer may be configured to measure a motion of the valve, thereby generating measurement data indicative of a volume of expressed milk. The sensor may further comprise a background motion accelerometer coupled to a portion of the housing, the background motion accelerometer configured to measure a motion of the apparatus. The motion of the apparatus may be subtracted from the motion of the valve to determine a normalized motion of the valve.

In some embodiments, the sensor comprises a beam-break sensor configured to detect passage of the expressed breast milk through the central channel. Measurements generated by the beam-break sensor may be used to determine a volume or a composition of the expressed breast milk.

In some embodiments, the sensor comprises an image sensor configured to detect passage of the expressed breast milk through the central channel. The image sensor may comprise a charge-coupled device (CCD) configured to count drops of the expressed breast milk passing through the central channel, thereby generating measurement data indicative of a volume of expressed milk.

In some embodiments, the sensor comprises a capacitive sensor configured to detect passage of the expressed breast milk through the central channel, thereby generating measurement data indicative of a volume of expressed milk.

In another aspect, a system for measuring expressed breast milk is provided. The system comprises a pumping device configured to express breast milk from one or more breasts, a collection vessel configured to receive the expressed breast milk, and a sensing adaptor configured to measure expressed breast milk. The sensing adaptor may be removably coupled to the pumping device on a first end and to the collection vessel on a second end. The sensing adaptor may comprise a sensor configured to measure one or more aspects of the expressed breast milk as the expressed breast milk passes from the pumping device through a central channel of the sensing adaptor into the collection vessel.

The system may further comprise a computing device in communication with the sensing adaptor, the computing device configured to receive measurement data generated by the sensor. The computing device may comprise a smartphone, a tablet, a desktop computer, or a laptop computer. The computing device may comprise a processing unit having instructions stored thereon for performing an analysis of the measurement data. The computing device may further comprise a user interface configured to display the measurement data to a user. The system may further comprise a remote server in communication with the sensing reservoir or the computing device, the remote server configured to perform one or more of analysis of the measurement data and storage of the measurement data.

In another aspect, a method for measuring expressed breast milk is provided. The method comprises providing a pumping device, a collection vessel, and a sensing adaptor having a first coupling mechanism on a first end configured to couple to the pumping device, and a second coupling mechanism on a second end configured to couple to the collection vessel. The method further comprises coupling the first coupling mechanism of the sensing adaptor to the pumping device, and coupling the second coupling mechanism of the sensing adaptor to the collection vessel. The method further comprises expressing breast milk from the breast using the pumping device and generating, via a sensor coupled to the sensing adaptor, measurement data indicative of one or more aspects of the expressed breast milk.

In some embodiments, the sensing adaptor further comprises a processing unit, and the method further comprises analyzing measurement data generated by the sensor with the processing unit. In some embodiments, the sensing adaptor further comprises a communication module, and the method further comprises transmitting measurement data generated by the sensor to one or more of a computing device or a server, via the communication module.

These and other embodiments are described in further detail in the following description related to the appended drawing figures.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a perspective view of a pumping device suitable for use with present embodiments;

FIGS. 2A and 2B illustrate an exemplary embodiment of a sensing adaptor;

FIG. 3 illustrates a top view of an exemplary embodiment of a processing unit suitable for incorporation with the sensing adaptor;

FIG. 4 illustrates an exemplary construction of a sensing adaptor;

FIG. 5A shows a cross-sectional view of an embodiment of a sensing adaptor comprising a strain gauge;

FIGS. 5B and 5C show detailed cross-sectional views of the embodiment shown in FIG. 5A;

FIG. 6 illustrates another exemplary embodiment of a sensing adaptor comprising a strain gauge;

FIG. 7 illustrates an exemplary embodiment of a sensing adaptor comprising an accelerometer;

FIG. 8 illustrates an exemplary embodiment of a sensing adaptor comprising a beam-break sensor;

FIG. 9 illustrates an exemplary embodiment of a sensing adaptor comprising an image sensor;

FIG. 10 illustrates an exemplary embodiment of a sensing adaptor comprising a capacitive sensor;

FIGS. 11A-11C illustrate exemplary computing device displays; and

FIGS. 12A-12B illustrate other exemplary displays.

### DETAILED DESCRIPTION OF THE INVENTION

Specific embodiments of the disclosed systems, devices, and methods will now be described with reference to the drawings. Nothing in this detailed description is intended to imply that any particular component, feature, or step is essential to the invention. Although the present invention primarily relates to breast milk, any description herein of expression and collection of breast milk can also be applied to other types of fluids expressed from the breast, such as colostrum. Furthermore, the disclosed embodiments may be used in other applications, particularly applications involving the measurement of any fluids collected in a collection vessel.

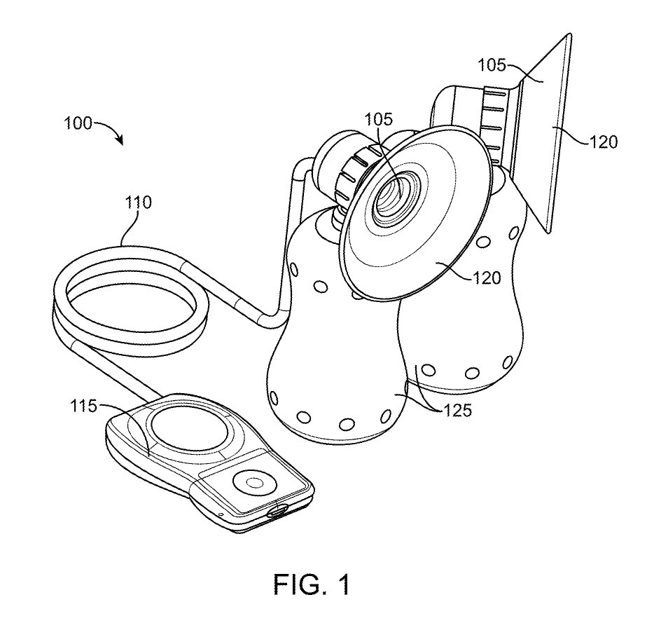

FIG. 1 illustrates an exemplary embodiment of a breast pump suitable for use with present embodiments. Pumping device 100 (also known as an “expression apparatus”) includes breast interfaces 105, a tube 110, and a controller 115 (sometimes also referred to as a “pendant unit”) operatively coupled to breast interfaces 105 through tube 110. Breast interfaces 105 include resilient and conformable flanges 120, for engaging and creating a fluid seal against the breasts, and collection vessels 125. Controller 115 houses the power source and drive mechanism for pumping device 100, and also contains hardware for various functions, such as controlling pumping device 100, milk production quantification, and communication with other devices, as described in further detail herein. Tube 110 transmits suitable energy inputs, such as mechanical energy inputs, from controller 115 over a long distance to breast interfaces 105. Breast interfaces 105 convert the energy inputs into vacuum pressure against the breasts in a highly efficient manner, resulting in the expression of milk into collection vessels 125. Power may be provided to the one or more sensors via a connection to the controller 115, or to another source of power.

A pumping device such as device 100 may further comprise or be coupled to one or more sensors configured to track various characteristics of the collected fluid. In many instances, it can be desirable to measure and track various characteristics of the collected fluid such as expressed breast milk, such as the amount of milk production (e.g., volume, weight), expression frequency (e.g., time, date), and/or expression duration. In existing approaches, the tracking of milk production is commonly accomplished by manual measurements and record-keeping. Sensors integrated for use with a pumping device can provide digital-based means to automatically measure and track milk production for improved convenience, efficiency, and accuracy. For example, sensors can be used to measure the volume of expressed milk as volume per unit time, or total volume per pumping session.

Sensors for quantifying the composition of the expressed milk may also be provided with a pumping device. The composition of breast milk can be valuable information for understanding whether an infant is obtaining the appropriate amount of nutrition via the milk. This information can help mothers or clinicians identify whether additional nutrition should be supplied to the infant, or whether the mother can make appropriate dietary or lifestyle changes to change the nutritional content of her milk. Components of breast milk considered to be nutritionally important include carbohydrates such as glucose and lactose, fats such as triglycerides, proteins such as lactoferrin, organic acids such as taurine, vitamins such as vitamin D, and minerals such as zinc, copper, and iron. Sensors may be provided for measuring the relative amounts of one or more of such components in the expressed milk. Sensors may also be configured to determine the estimated caloric value of the expressed milk and/or the percentage of alcohol present in the milk. Such sensors may include devices that can spectroscopically measure the presence of certain compounds in a volume of breast milk, or devices that can measure the enzymatic activity produced by certain compounds of breast milk that act as substrates for specific enzymes.

The one or more sensors may be coupled to a pumping device, such as to one or more portions of a breast interface. Alternatively or in combination, one or more sensors may be coupled to a collection vessel. In embodiments in which the one or more sensors are coupled to one or more portions of the breast interface or to a collection vessel, the sensors may be further coupled to the pump controller via one or more communication lines configured to transmit signals between the sensors and the controller. Alternatively, the sensors may be coupled wirelessly to the controller or to a mobile device configured to control the pumping device, for example via a communication module coupled to the sensors. Embodiments of pumping devices and collection vessels having one or more sensors coupled thereto are disclosed in greater detail in U.S. patent application Ser. No. 14/616,557 (Attorney Docket No. 44936-704.201), the entire content of which is incorporated herein by reference.

The one or more sensors to track characteristics of the collected fluid may also be provided in a separate accessory adaptable for use with various pumping devices, such as a sensing adaptor as described herein. Providing a sensing capability in an accessory or adaptor completely separate from the pumping device can have many benefits for users. The sensing adaptor may be adaptable for use with various pumping devices, including many commercially available systems, providing a great range of flexibility for users. For example, a user may choose to add the sensing adaptor to a pumping system she already owns, in order to gain the benefits provided by an automatic sensing function. In addition, in case of failure of one or more of its components, a stand-alone sensing adaptor may be easier to repair or replace than a sensor integrated into a pumping system.

The sensing adaptors described herein are configured to couple to a pumping device on one end, and to a collection vessel on the other end. For example, the sensing adaptor may couple to a breast interface or flange of a breast pump on one end, and couple to a collection bottle or bag for the expressed breast milk on the other end. The sensing adaptors as described herein can measure milk expression in real time as the milk is being expressed by a pumping device and collected into a collection vessel. The sensing adaptors can include one or more sensors for generating measurement data indicative of one or more characteristics of milk expression as described herein (e.g., volume of expressed milk, composition of the expressed milk, etc.). Any description herein pertaining to measurement of volume can also be applied to measurements of other characteristics, and vice-versa. Any suitable type of sensor can be used, such as accelerometers, Hall effect sensors, photodiode/LED sensors, CCD sensors, cameras and other imaging devices, capacitive sensors, strain gauges, etc., and such sensors can be used in any number and combination. The sensors can be positioned at any location on the adaptor suitable for monitoring fluid flow from the pumping device.

A processing unit may be suitably combined with any sensing adaptor described herein, wherein the processing unit may be configured to receive data from the sensor and store the data, analyze the data, and/or transmit the data to another device. A sensing adaptor having an integrated sensor and processing unit can help automate the management and monitoring of milk production, thus reducing the need for manually maintaining records related to milk production. For example, a sensing adaptor can monitor the quantity of milk produced, and automatically process and send the data to a computing device, from which the user may easily access the information. Such a system can greatly improve convenience for the users, and also help reduce human errors related to manual record maintenance.

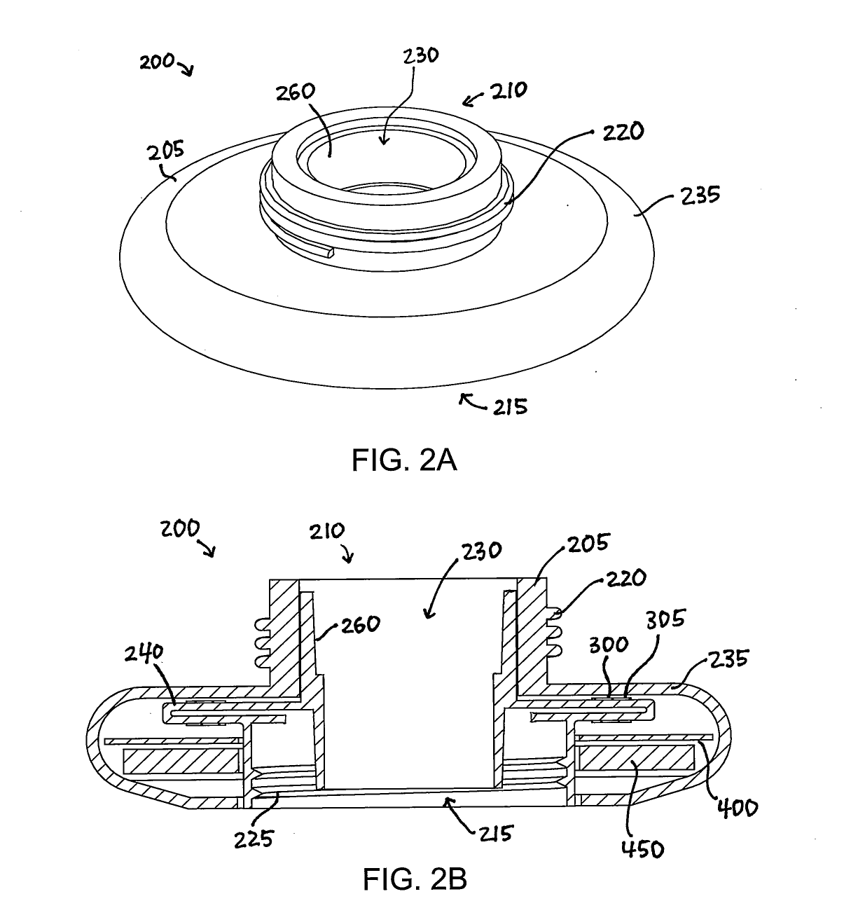

FIGS. 2A and 2B illustrate an exemplary embodiment of a sensing adaptor 200. FIG. 2A shows a perspective view of the sensing adaptor, while FIG. 2B shows a cross sectional view of the adaptor. The sensing adaptor comprises a housing 205 having a first end 210 that interfaces with a pumping device, and a second end 215 that interfaces with a collection vessel. A first coupling mechanism 220 is disposed adjacent the first end, the first coupling mechanism configured to removably couple to a portion of the pumping device (e.g., breast interface, flange or flange body). A second coupling mechanism 225 is disposed adjacent the second end, the second coupling mechanism configured to removably couple to the opening of the collection vessel. The housing further comprises a channel member 260 defining a central channel 230 extending between the first end and the second end, wherein the central channel permits passage of the fluid from the pumping device into the collection vessel.

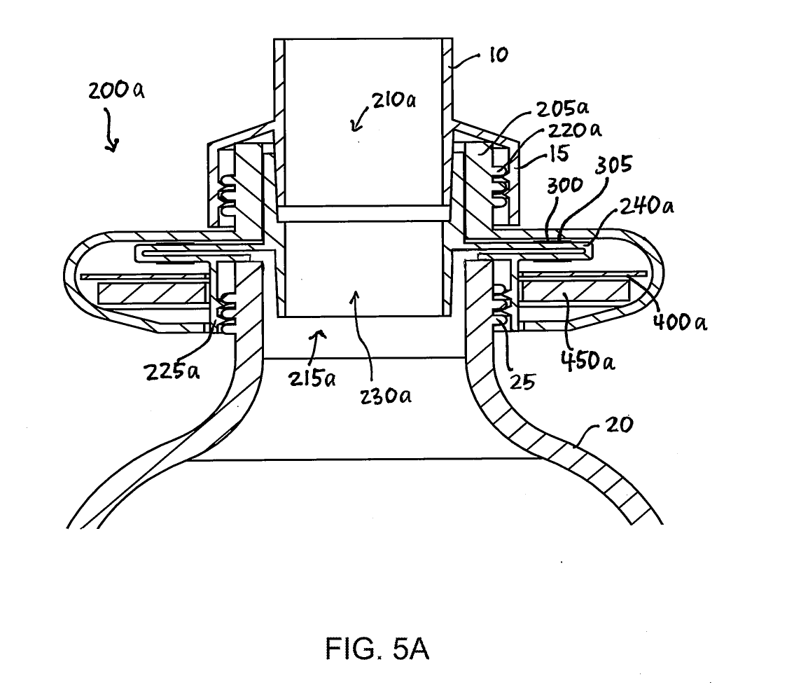

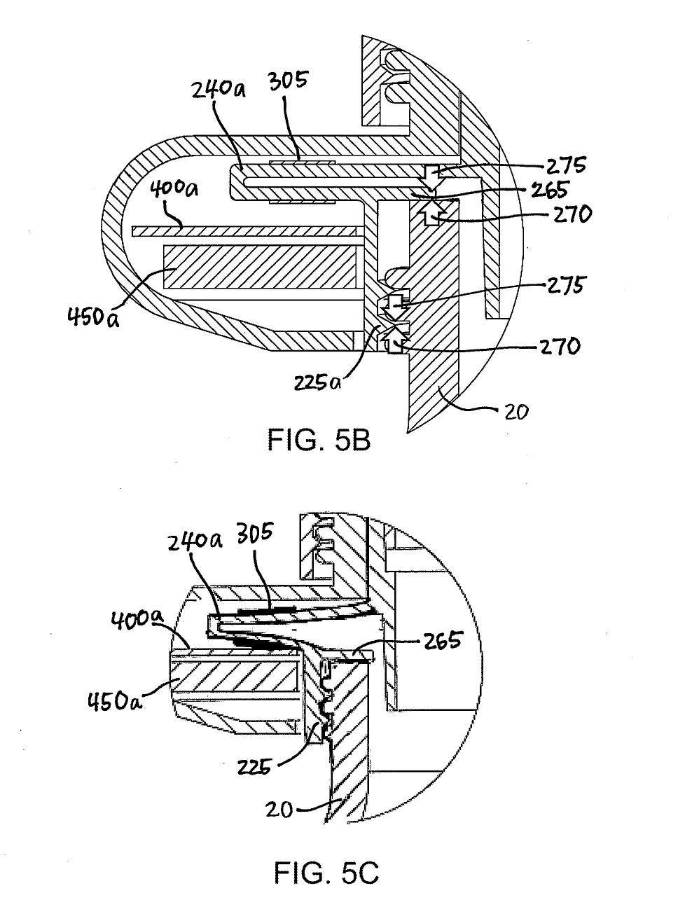

The first coupling mechanism and the second coupling mechanism may comprise any coupling mechanisms known in the art, such as screw threads, quarter turn couplings, bayonet couplings, interference fits, snap fits, press fits, collets, and the like. Some embodiments of the sensing adaptor may require specific types of coupling mechanisms in order for the sensor mechanism to function properly; for example, in the embodiment illustrated in FIGS. 5A-5C, screw threads or quarter turn couplings may be particularly well-suited. In such cases, the coupling mechanisms most compatible with the sensor mechanism may be selected. In preferred embodiments, the first coupling mechanism comprises male screw threads, and the second coupling mechanism comprises female screw threads, so as to make the sensing adaptor widely adaptable for use with many off-the-shelf pumping devices utilizing screw threads to attach a collection vessel to the pumping device.

As shown in FIG. 2B, the adaptor 200 may comprise one or more sensors 300 configured to measure one or more characteristics of the expressed breast milk. While FIG. 2B shows a plurality of sensors located on an interior lateral member 240 of the housing, the sensors may be provided in any number and at any location on the sensing adaptor. Types and configurations of sensors suitable for incorporation with the sensor adapter are described in further detail herein with respect to specific exemplary embodiments. The sensing adaptor may further comprise a processing unit 400 in communication with the sensor, the processing unit configured to receive data from the sensor and store the data, analyze the data, and/or transmit the data to another device. The processing unit of a sensing adaptor may perform analysis of the collected data and transmit the analyzed data to another device; alternatively, the processing unit may transmit raw sensor data to a another computing device, and the computing device may be used to analyze the data. The processing unit and/or the sensor may be powered by a power source 450, such as a battery, operatively coupled to the processing unit. The housing may comprise a shroud 235 that surrounds the sensor and/or other electrical components such as the processing unit and the power source, in order to protect the sensor and other components. The shroud may comprise a material with properties such that the shroud can physically protect the encased structures from external forces, for example forces applied by a user as the user attaches the sensing adaptor to a pumping device or to a collection vessel. The shroud may completely encase the sensor and/or other components in a leak-proof manner to protect the encased structures from water damage, such that the sensing adaptor may be washed and reused.

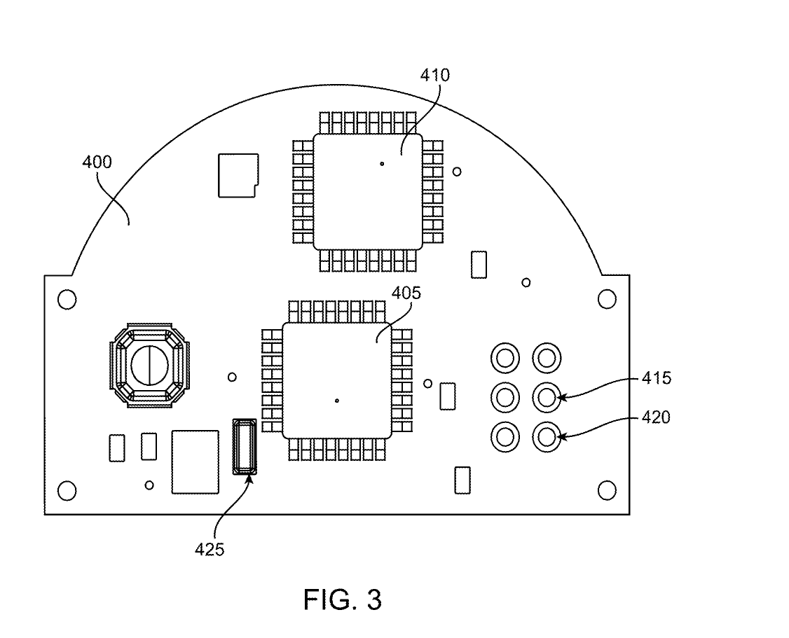

FIG. 3 illustrates an exemplary embodiment of a processing unit 400 suitable for incorporation with the sensing adaptor. The processing unit may comprise one or more of a printed circuit board (PCB) housing one or more of a microcontroller 405, a communication module 410, a sensor connection 415, a power connection 420, and a timer 425. The processing unit may receive signals from the sensor through the sensor connection 415, and the signals may be transmitted to the microcontroller 405. The microcontroller may comprise a non-transitory computer readable medium comprising instructions to collect and process the signals received from the sensor. The microcontroller may further comprise instructions to transmit the collected and/or processed signals to the communication module 410. The communication module may comprise a wireless transmitter/receiver such as a Blue Tooth module, for example. The communication module may be configured to transmit the sensor data to another computing device, such as a mobile phone, tablet, or personal computer, and/or to a server, for data analysis and/or display. Power may be supplied to the processing unit via a power source comprising a battery or a direct contact connection such as a cable or pad connectors. Alternatively or in combination, power may be supplied via an inductive charging system comprising a battery and a wireless charger, which may be charged using an inductive charging method as known in the art.

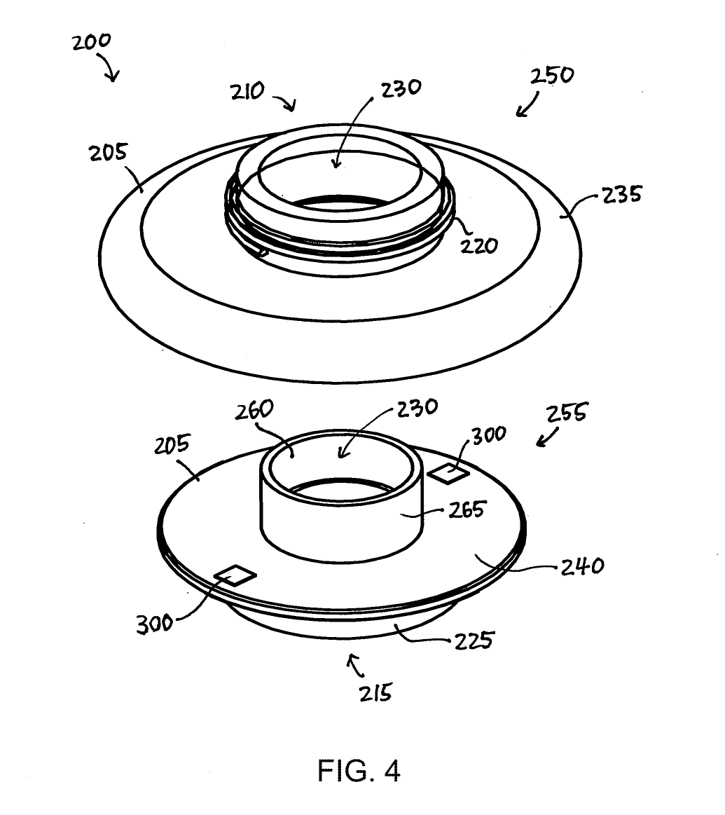

FIG. 4 illustrates an exemplary construction of a sensing adaptor. The sensing adaptor 200 may comprise an exterior portion 250 and an interior portion 255, coupled together to form the housing of the sensing adaptor. The exterior portion can include the first coupling mechanism 220 configured to couple to a pumping device, and the shroud 235 configured to encase and protect one or more components of the sensing adaptor. The interior portion can include the second coupling mechanism 225 configured to couple to a collection vessel, and the channel member 260 defining the central channel 230. The interior portion may support one or more sensors 300 configured to measure a characteristic of the expressed breast milk as the milk flows through the central channel 230. The interior portion may further comprise one or more lateral members 240 configured to support the one or more sensors, wherein the lateral member may comprise an annular shape as shown in FIG. 4, or any other suitable shape such as one or more elongate beams. Alternatively, the sensors may be supported at any other appropriate location of the interior portion, as described in further detail herein. For example, the interior portion may further comprise a flap valve coupled to the channel member, and the sensor may be coupled to the flap valve.

A protruding portion 265 of the channel member may couple to an interior surface of the first coupling mechanism 220, in order to couple the exterior and interior portions together. Further, a bottom portion of the shroud may couple to an exterior surface of the second coupling mechanism 225. Preferably, the exterior and interior portions are coupled together tightly such that the two portions are in substantially fixed positions relative to one another during the use of the sensing adaptor. For example, the exterior and interior portions may be permanently coupled together using adhesives (e.g., epoxy or cyanoacrylate adhesives) or via ultrasonic welding. Alternatively, the exterior and interior portions may be removably coupled using fastening members (e.g., pins or screws) or via one of many fastening mechanisms known in the art (e.g., threadings, snap fits, etc.). Preferably, the exterior and interior portions are coupled together to form a fluid tight seal, such that the sensing adaptor may be exposed to fluids (e.g., during milk expression or during washing of the adaptor) without adversely affecting the function of the structures encased within the shroud, such as the sensor or of one or more electrical components operatively coupled to the sensor.

In some embodiments, the sensor of the sensing adaptor may comprise one or more strain gauges configured to measure the volume of expressed fluid. The strain gauges can be situated at any suitable position in the sensing adaptor. For example, a strain gauge can be coupled to an interior lateral member of the housing, wherein the lateral member is coupled to the second coupling mechanism, such that the strain gauge can measure the strain placed on the second coupling mechanism by the collection vessel coupled thereto. Alternatively or in combination, a strain gauge can be coupled to a flap valve (or any other valve permitting passage of expressed fluid) and configured to determine the volume based on the displacement of the valve over time. Embodiments of sensors comprising strain gauges are disclosed in greater detail in U.S. patent application Ser. No. 14/616,557 (Attorney Docket No. 44936-704.201), which has previously been incorporated by reference.

FIG. 5A shows a cross-sectional view of an embodiment of a sensing adaptor 200a comprising a strain gauge. As described herein, the sensing adaptor comprises a housing 205a having a first end 210a, second end 215a, and a central channel 230a defined therethrough. The first coupling mechanism 220a is configured to couple to a portion 15 of a pumping device 10 producing the expressed fluid. The pumping device 10 may comprise any pumping device configured to express fluid, such as any breast milk expression device. The second coupling mechanism 225a is configured to couple to a collection vessel 20 configured to receive the expressed fluid, for example by threadably engaging a threaded opening 25 of the collection vessel. The collection vessel 20 may comprise any collection vessel or reservoir suitable for coupling to any pumping device 10. The adaptor further comprises sensors 300a, the sensors comprising strain gauges 305 coupled to the interior lateral member 240a of the adaptor housing.

FIG. 5B shows a detailed cross-sectional view of the embodiment shown in FIG. 5A. The lateral member 240a supporting the strain gauges 305 is attached to the second coupling mechanism 225a, such that the loads placed on the second coupling mechanism by the collection vessel 20 are transmitted to the lateral member. Further, the lateral member is configured such that at least a portion of the lateral member undergoes deformation increasingly as the weight of the collection vessel coupled thereto increases. Preferably, the sensing adaptor is configured such that the load exerted by the collection vessel is completely isolated and transmitted to the second coupling mechanism. In order to achieve a complete isolation of the loads measured by the strain gauges, a lip 265 may be provided, such that the top surface of the collection vessel 20 bottoms out on, or becomes flush with, the lip when the vessel is coupled to the sensing adaptor. The lip 265 is connected to the second coupling mechanism 225a and the lateral member 240a, such that the forces 270 exerted by the collection vessel are countered by the reaction forces 275 of the sensing adaptor. Such a configuration can allow an effective isolation of the loads exerted by the collection vessel. In embodiments in which the sensing adaptor is constructed in two or more portions coupled together, the portions may be coupled in a configuration that appropriately transmits loads to the second coupling mechanism. For example, in the embodiment shown in FIG. 4 wherein the sensing adaptor comprises an exterior portion and an interior portion coupled together, the two portions may be coupled such that the bottoms of the two portions are fixed in position relative to one another (i.e., are not free to translate relative to one another), to ensure that the load of the collection vessel is completely absorbed by the interior portion.

FIG. 5C shows a detailed cross-sectional view of the embodiment shown in FIG. 5A, wherein the lateral member 240a is under load. As milk is expressed and begins to collect in the collection vessel 20, the weight of the collection vessel increases accordingly. The increasing weight exerts an increasing downward load on the second coupling mechanism 225a coupled to the collection vessel. The increasing downward load on the second coupling mechanism can cause the lateral member 240a supporting the strain gauges 305 to bend under the moment created by the load, and deflect as shown in FIG. 5C. The strain gauges may comprise one of many configurations known in the art, such as a Wheatstone bridge, that can measure the strain in the lateral member. An electrical circuit can be created that interrogates the resistance of the strain gauges, and the change in resistance can be used to determine the weight driving the deformation. The change in weight of the collection vessel can then be used to determine the weight and estimated volume of the expressed milk collected in the collection vessel. For example, the weight of the expressed milk can be translated to a volume of milk based on a calibration curve stored on a database.

The data collected by the strain gauges may be received and/or analyzed by the processing unit 400a in communication with the strain gauges. Power may be supplied to the processing unit and/or the strain gauges via a power source 450a. The processing unit may further transmit the sensor data to another computing device via a communication module. The user may view and track the sensor data from the computing device, for example via a mobile application on a mobile phone. The sensing adaptor embodiment of FIGS. 5A-5B may be susceptible to external forces exerted on the collection vessel, such as the vessel being placed on a surface or being held by a user, since such forces can affect accurate sensing of the collection vessel weight. Hence, this sensing adaptor embodiment is preferably used with hands-free pumping systems, wherein the collection vessel is configured to be suspended freely in a vertical position without the user holding or touching any portions of the collection vessel during pumping.

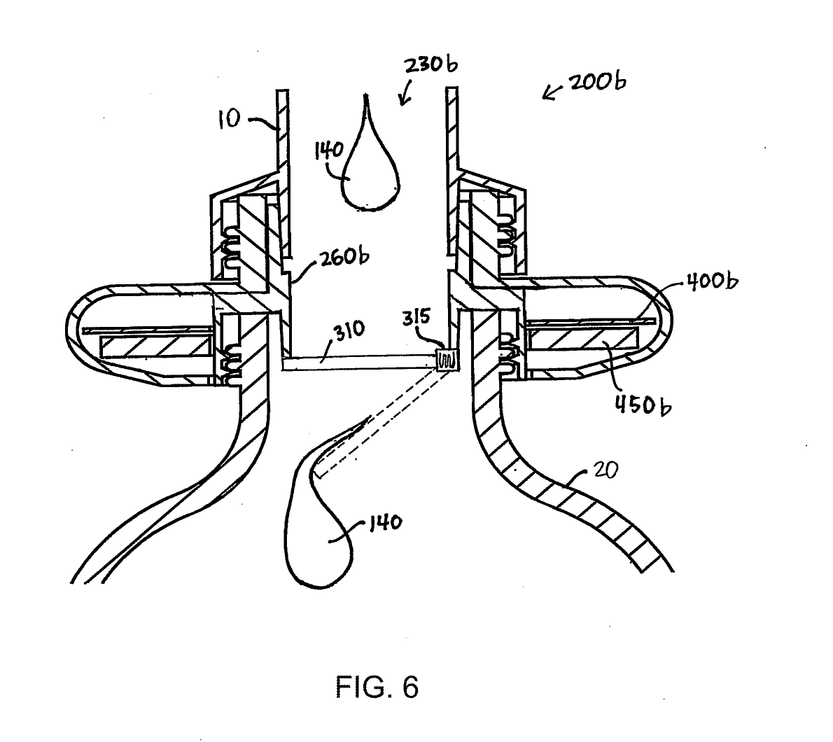

FIG. 6 illustrates another exemplary embodiment of a sensing adaptor 200b comprising a strain gauge 315. The sensing adaptor 200b may be configured to couple to a pumping device 10, wherein the pumping device 10 may comprise any pumping device configured to express fluid, such as any breast milk expression device. The sensing adaptor 200b may be further configured to couple to a collection vessel 20, wherein the collection vessel 20 may comprise any collection vessel or reservoir suitable for coupling to any pumping device 10. The sensing adaptor 200b comprises a valve 310 coupled to channel member 260b. The valve 310 may comprise a flap valve, a duckbill valve, or any other valve configured to open in response to pressure or weight exerted by the fluid accumulated on top of the valve. The valve is configured to block the central channel 230b when the valve is in not under load, such that as milk is expressed, the expressed milk 140 accumulates in the central channel, on top of the valve. Eventually, the weight of the accumulated milk is sufficient to actuate and open the valve. A strain gauge 315 is coupled to the valve, such that the strain gauge can measure the strain exerted by the movement of the valve as the valve opens. The movement of the valve may in turn be correlated to the amount of collected fluid. The strain gauge may be operatively coupled to a processing unit 400b of the sensing adaptor, and may transmit the collected data to the processing unit for further processing, storage, and/or transmission to another computer device as described herein. A power source 450b may be supply power to the processing unit and/or the strain gauge sensor.

In some embodiments, the sensor of the sensing adaptor may comprise one or more accelerometers configured to measure the volume of expressed fluid. The accelerometers may measure a position and/or motion of a valve, and the resultant measurement data can be interrogated to quantify the fluid flow. In some instances, movements of the user may cause the accelerometers to produce motion signals that are erroneously interpreted as valve motion. Accordingly, in preferred embodiments, suitable approaches are used to distinguish between signals resulting from motion of the user and signals generated by motion of the valves. For example, the sensing adaptor may comprise a background motion accelerometer in addition to a valve accelerometer, wherein the background motion accelerometer is configured to measure background motion including motion of a user, as described in further detail herein. The background motion measured by the background motion accelerometer may be subtracted from the motion measured by the valve accelerometer, in order to obtain the isolated valve motion. Embodiments of sensors comprising accelerometers are disclosed in greater detail in U.S. patent application Ser. No. 14/616,557 (Attorney Docket No. 44936-704.201), which has previously been incorporated by reference.

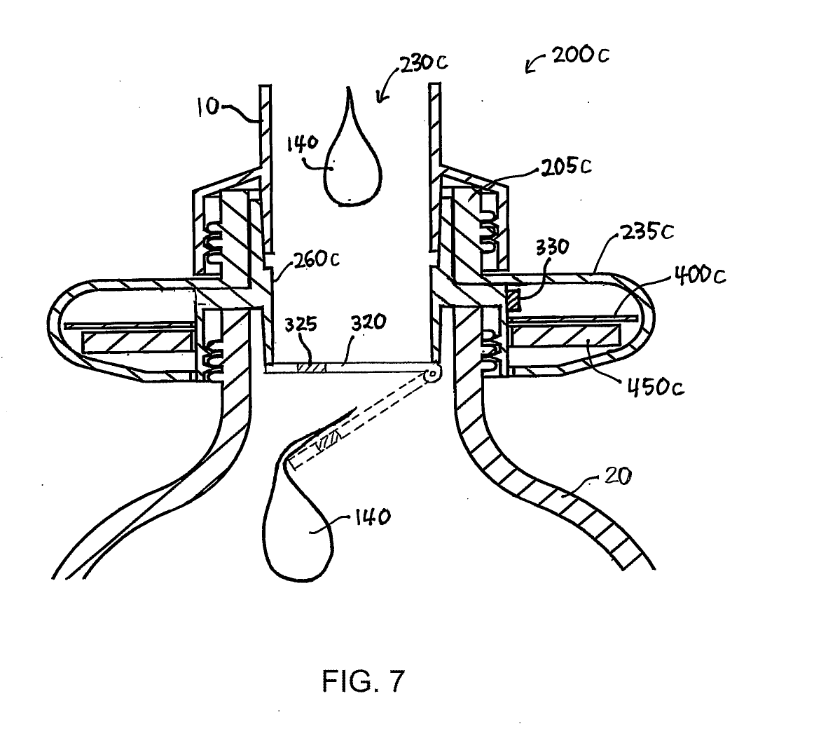

FIG. 7 illustrates an exemplary embodiment of a sensing adaptor 200c comprising an accelerometer 325. The sensing adaptor 200c may be configured to couple to a pumping device 10, wherein the pumping device 10 may comprise any pumping device configured to express fluid, such as any breast milk expression device. The sensing adaptor 200c may be further configured to couple to a collection vessel 20, wherein the collection vessel 20 may comprise any collection vessel or reservoir suitable for coupling to any pumping device 10. The sensing adaptor 200c can comprise a valve 320 coupled to channel member 260c. A valve accelerometer 325 may be coupled to the valve. The valve 320 may comprise a flap valve, a duckbill valve, or any other valve configured to open in response to pressure or weight exerted by the fluid accumulated on top of the valve. The valve is configured to block the central channel 230c when the valve is in not under load, such that as milk is expressed, the expressed milk 140 accumulates in the central channel, on top of the valve. Eventually, the weight of the accumulated milk is sufficient to actuate and open the valve. Movement of the valve is tracked using the accelerometer coupled thereto, and data from the accelerometer can then be transmitted to a processing unit 400c for further processing, storage, and/or transmission to another computer device as described herein. A power source 450c may be supply power to the processing unit and/or the strain gauge sensor.

Optionally, the sensing adaptor 200c may further comprise a background motion accelerometer 330 configured to measure motion of the system unrelated to the motion of the valve 320. The background motion accelerometer may be coupled to a portion of the housing 205c whose movement is not affected by the motion of the valve 320. The background motion accelerometer is preferably coupled to an interior portion of the housing such that it is protected from external forces by the shroud 235c. The background motion accelerometer 330 and the valve accelerometer 325 may both be coupled to a processing device 400c and/or a power source 450c disposed within the sensing adaptor. The valve motion accelerometer may often measure background motion unrelated to the motion of the valve 320, such as user motion, in addition to the motion of the valve. In order to normalize the measurement of the valve motion accelerometer against the background motion, the background motion accelerometer can be configured to track the overall movement of the sensing adaptor in space, so as to measure the background motion of the adaptor unrelated to the motion of the valve. The signal measured by the background motion accelerometer can then be subtracted from the signal measured by the valve accelerometer, to exclude or minimize the contribution of background motion to valve motion. Data generated by the accelerometers 325 and 330 may be transmitted to a processing unit 400c for further processing, storage, and/or transmission to another computer device as described herein. Signal processing may be performed the processing unit 400c integrated with the sensing adaptor 200c, or raw data from the two accelerometers 325 and 330 may be transmitted to another computing device, where the signal processing may be performed.

In other exemplary embodiments, the sensing adaptor can utilize one or more beam-break sensors (e.g., infrared-based, laser-based, etc.) situated adjacent the central channel of the adaptor. The beam-break sensor can include a plurality of sensor components and can be configured to detect passage of fluid between or near one or more of the components. Preferably, the sensor can be configured to generate a signal when the expressed fluid breaks a beam by passing between a beam emitter and a beam detector. The resultant signal can be used to produce measurement data indicative of the volume of expressed fluid. For example, the measurement data can be based on the length of time the fluid passes between or near the sensor components. Embodiments of sensors comprising beam-break sensors are disclosed in greater detail in U.S. patent application Ser. No. 14/616,557 (Attorney Docket No. 44936-704.201), which has previously been incorporated by reference.

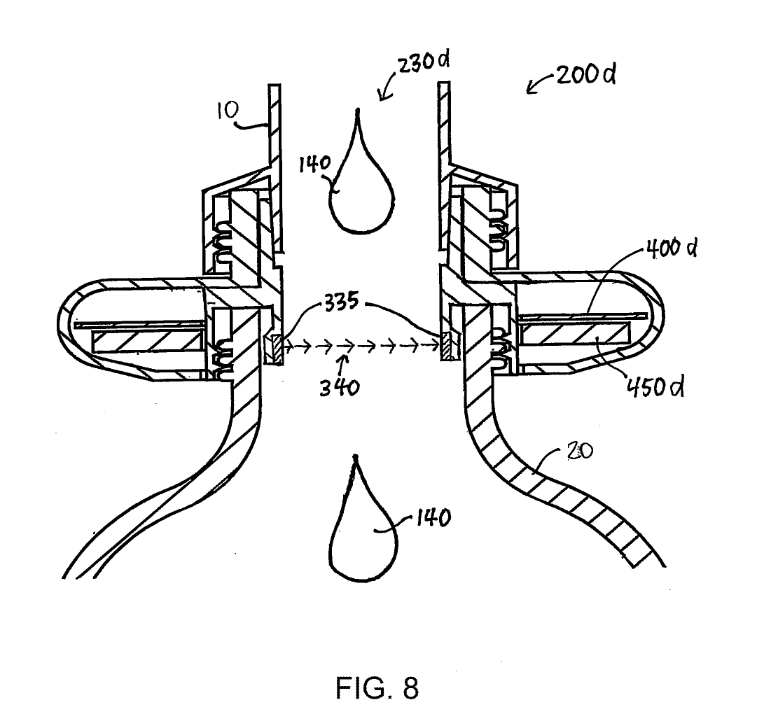

FIG. 8 illustrates an exemplary embodiment of a sensing adaptor 200d comprising a beam-break sensor 335. The sensing adaptor 200d may be configured to couple to a pumping device 10, wherein the pumping device 10 may comprise any pumping device configured to express fluid, such as any breast milk expression device. The sensing adaptor 200d may be further configured to couple to a collection vessel 20, wherein the collection vessel 20 may comprise any collection vessel or reservoir suitable for coupling to any pumping device 10. The sensing adaptor 200d comprises a beam-break sensor 335, disposed adjacent the central channel 230d. As droplets of expressed milk 140 drain from the pumping device 10 into the central channel, they can break the light beam 340, allowing measurement of the fluid expressed. The data from the sensor can then be transmitted to a processing unit 400d for further processing, storage, and/or transmission to another computer device as described herein. A power source 450d may be supply power to the processing unit and/or the strain gauge sensor.

Beam-break sensors may also be adapted to measure a composition of the expressed milk. For example, a beam-break sensor may comprise a beam emitter configured to emit light in a wavelength range known to be absorbed by a specific component in breast milk. The beam-break sensor may further comprise a beam detector configured to detect light reflected from or transmitted through a droplet of expressed milk. The beam detector can hence measure the amount of light absorbed by the droplet of milk, and this information can be used to estimate the amount of the specific component in the expressed milk.

In other exemplary embodiments, the pumping devices described herein can include one or more image sensors for capturing images of the fluid in order to quantify the expression volume. The image sensors may comprise a charge-coupled device (CCD), active pixel sensors in complementary metal-oxide-semiconductor (CMOS), a camera, or a combination thereof. The image sensors may be integrated with or coupled to a suitable portion of the sensing adaptor. In exemplary embodiments, the sensing adaptor includes a valve permitting the passage of expressed fluid, as previously described herein, and a suitable image sensor is positioned on or near the valve in order to capture images of fluid passing through the valve. Preferably, the image sensor is operably coupled to a processing unit configured to analyze the image data (e.g., using a suitable image analysis algorithm) in order to determine the fluid volume. For example, the image sensors can be used to capture images of drops of fluid, and the images can be analyzed to count the number of drops. In some instances, the image data can be transmitted to a computing device (e.g., a smartphone) for analysis, as described in further detail below. Embodiments of sensors comprising image sensors are disclosed in greater detail in U.S. patent application Ser. No. 14/616,557 (Attorney Docket No. 44936-704.201), which has previously been incorporated by reference.

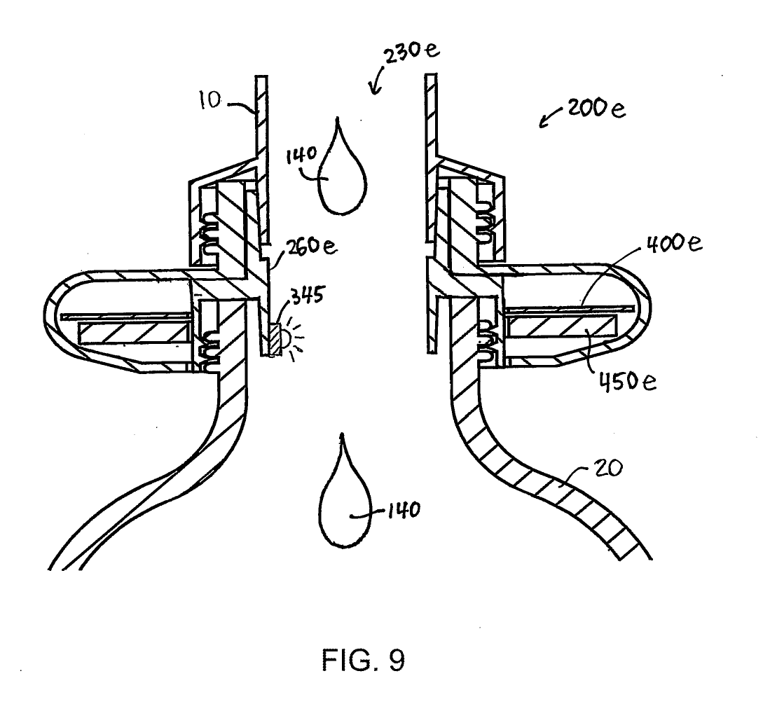

FIG. 9 illustrates an exemplary embodiment of a sensing adaptor 200e comprising an image sensor. The sensing adaptor 200e may be configured to couple to a pumping device 10, wherein the pumping device 10 may comprise any pumping device configured to express fluid, such as any breast milk expression device. The sensing adaptor 200e may be further configured to couple to a collection vessel 20, wherein the collection vessel 20 may comprise any collection vessel or reservoir suitable for coupling to any pumping device 10. The sensing adaptor 200e comprises a CCD or CMOS device 345 coupled to the channel member 260e, adjacent the central channel 230e. As milk is expressed by the pumping device 10, the expressed milk 140 passes through the image sensor 345 which detects the fluid and allows quantification thereof as previously described. Data from the sensor can then be transmitted to a processing unit 400e for further processing, storage, and/or transmission to another computer device as described herein. A power source 450e may be supply power to the processing unit and/or the strain gauge sensor.

In some exemplary embodiments, the sensing adaptors described herein can employ one or more capacitive sensors for measuring fluid volume. The capacitive sensors can include a plurality of sensor components and can be configured to detect passage of fluid between or near one or more of the components. Preferably, the sensors can be configured to generate a signal when the expressed fluid changes the capacitance between the surfaces of the sensor components. The resultant signal can be used to produce measurement data indicative of the volume of expressed fluid. For example, the measurement data can be based on the length of time the fluid passes between or near the sensor components. Embodiments of sensors comprising capacitive sensors are disclosed in greater detail in U.S. patent application Ser. No. 14/616,557 (Attorney Docket No. 44936-704.201), which has previously been incorporated by reference.

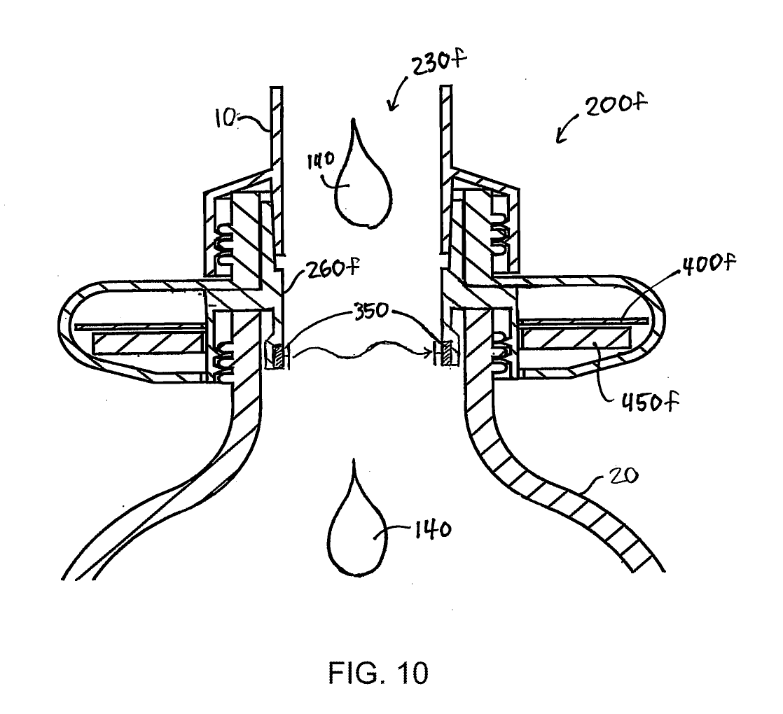

FIG. 10 illustrates an exemplary embodiment of a sensing adaptor 200f comprising a capacitive sensor 350. The sensing adaptor 200f may be configured to couple to a pumping device 10, wherein the pumping device 10 may comprise any pumping device configured to express fluid, such as any breast milk expression device. The sensing adaptor 200f may be further configured to couple to a collection vessel 20, wherein the collection vessel 20 may comprise any collection vessel or reservoir suitable for coupling to any pumping device 10. The sensing adaptor 200f comprises a capacitive sensor 350 coupled to the channel member 260f adjacent the central channel 230f. As milk is expressed by the pumping device 10, the expressed milk 140 passes through the capacitive sensor, which can sense the passage of the fluid and/or estimate the volume of the fluid passing through based on the change in capacitance between the components of the sensor. The data from the sensor can then be transmitted to a processing unit 400f for further processing, storage, and/or transmission to another computer device as described herein. A power source 450f may be supply power to the processing unit and/or the strain gauge sensor.

In any of the embodiments disclosed herein, the sensing adaptors described herein can be configured to communicate with another entity, such as one or more computing devices and/or servers. Exemplary computing devices include personal computers, laptops, tablets, and mobile devices (e.g., smartphones, cellular phones). The servers can be implemented across physical hardware, virtualized computing resources (e.g., virtual machines), or any suitable combination thereof. For example, the servers may comprise distributed computing servers (also known as cloud servers) utilizing any suitable combination of public and/or private distributed computing resources. The computing devices and/or servers may be in close proximity to the sensing adaptor and the pumping device (short range communication), or may be situated remotely from the sensing adaptor and the pumping device (long range communication). Any description herein relating to communication between a computing device and a sensing adaptor can also be applied to communication between a server and a sensing adaptor, and vice-versa.

The sensing adaptor can communicate with another computing device via a communication module, as described herein. The communication module can utilize any communication method suitable for transmitting data, such as a wired communication (e.g., wires, cables such as USB cables, fiber optics) and/or wireless communication (Bluetooth®, WiFi, near field communication). In many embodiments, data can be transmitted over one or more networks, such as local area networks (LANs), wide area networks (WANs), telecommunications networks, the Internet, or suitable combinations thereof.

The computing device may be associated with data stores for storage of the measurement data and/or analysis results. Applications of the computing device can also collect and aggregate the measurement data and/or analysis results and display them in a suitable format to a user (e.g., charts, tables, graphs, images, etc.). Preferably, the application includes additional features that allow the user to overlay information such as lifestyle choices, diet, and strategies for increasing milk production, in order to facilitate the comparison of such information with milk production statistics. The analysis and display functionalities described herein may be performed by a single entity, or by any suitable combination of entities. For example, in many embodiments, data analysis can be carried out by a server, and the analysis results may be transmitted to another computing device for display to the user.

Other types of data can also be transmitted between the sensing adaptor and other computing devices. For example, in many embodiments, firmware updates for one or more components of the sensing adaptor can be transmitted to the adaptor from the computing device.

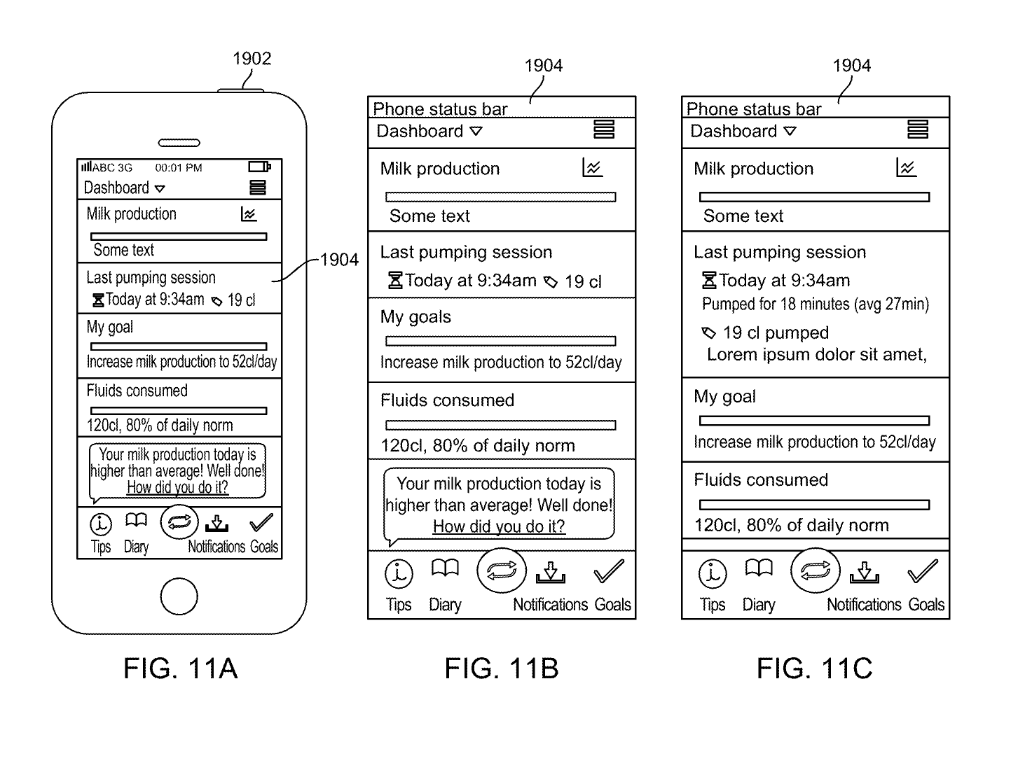

FIGS. 11A-11C illustrate exemplary computing device displays 1904. For example, FIG. 11A illustrates an exemplary display on a mobile phone 1902 and graphically illustrates milk production, the time of the last pumping session, a graphic of goal attainment, and a graphic illustrating the fluid consumption of the user. Additionally, the display 1904 may also provide user encouragement or user feedback based on the amount of milk production. FIG. 11B is an enlarged view of the display 1904 in FIG. 11A. FIG. 11C illustrates additional information that the display 1904 may show when a touch screen is actuated (e.g. by swiping or touching the screen). For example, the volume of the milk expressed is indicated after the “Last Pumping Session” section of the display is selected. Some or all items may be expanded, as also indicated in FIG. 11C. Additional information, or in some situations, less information may be displayed as desired.

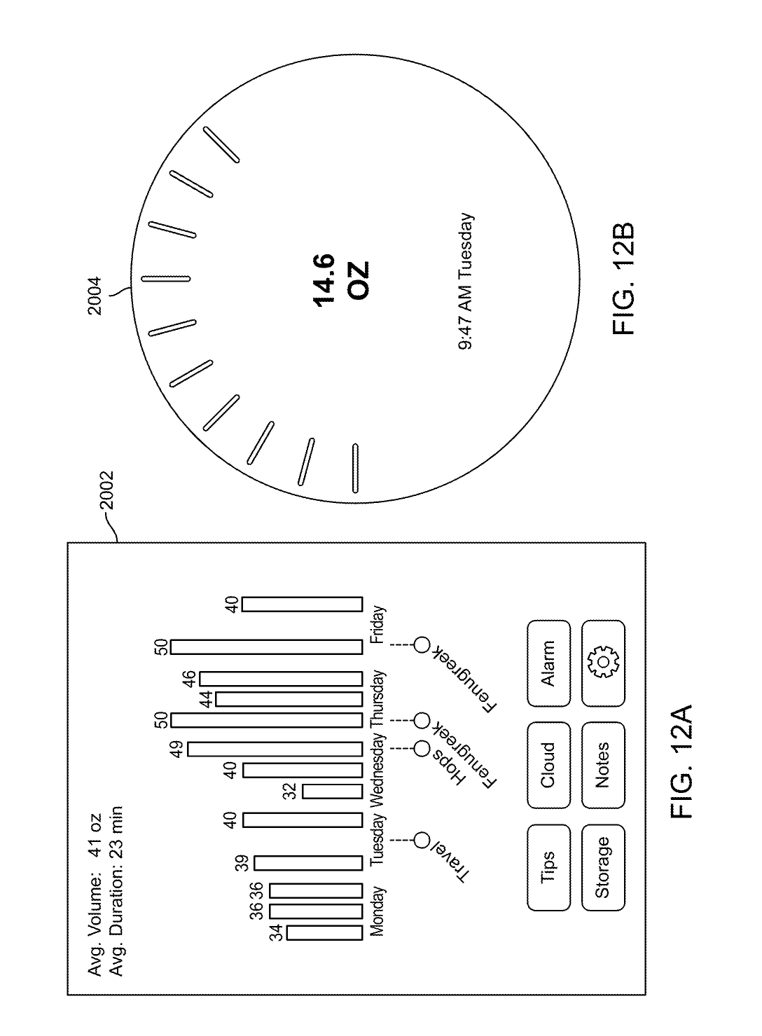

FIGS. 12A-12B illustrate other exemplary displays which may be used in a milk expression system. For example, FIG. 12A is an exemplary display 2002 on any of the computing devices disclosed herein and operably coupled with any of the pump units described herein. The display may indicate an average volume of milk expressed over any time period, along with an average duration of the expression session during that same time period. Graphics may be used (e.g. bar chart, pie chart, x-y plot, etc.) to show volume expressed during individual sessions over the course of several days, here Monday through Friday. The display may allow a user to annotate the display so that missed sessions may be accounted for, for example if a session is omitted due to traveling, the display may show travel during that time period. Other annotations may also be made, such as when certain foods or nutritional supplements are taken, here hops or fenugreek. This allows the user to recall when expressed milk samples were obtained relative to the consumption of the food or nutritional supplements. The display may have other functional buttons such as for obtaining tips, accessing the cloud, setting an alarm, making notes, storing data, or establishing system preferences. FIG. 12B illustrates another exemplary display 2004 of the computing device. The display 2004 is similar to a dashboard style gauge and indicates the volume of fluid expressed and collected and the time. Other information may also be displayed.

The various techniques described herein may be partially or fully implemented using code that is storable upon storage media and computer readable media, and executable by one or more processors of a computer system. Storage media and computer readable media for containing code, or portions of code, can include any appropriate media known or used in the art, including storage media and communication media, such as but not limited to volatile and non-volatile, removable and non-removable media implemented in any method or technology for storage and/or transmission of information such as computer readable instructions, data structures, program modules, or other data, including RAM, ROM, EEPROM, flash memory or other memory technology, CD-ROM, digital versatile disk (DVD) or other optical storage, magnetic cassettes, magnetic tape, magnetic disk storage or other magnetic storage devices, solid state drives (SSD) or other solid state storage devices, or any other medium which can be used to store the desired information and which can be accessed by the a system device. Based on the disclosure and teachings provided herein, a person of ordinary skill in the art will appreciate other ways and/or methods to implement the various embodiments.

It shall be understood that different aspects of the invention can be appreciated individually, collectively, or in combination with each other. Suitable elements or features of any of the embodiments described herein can be combined or substituted with elements or features of any other embodiment.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An apparatus for measuring expressed breast milk, the apparatus comprising:
a housing having a first end, a second end, and a central channel defined therethrough;
a first coupling mechanism disposed adjacent the first end, the first coupling mechanism configured to removably couple to a pumping device;
a second coupling mechanism disposed adjacent the second end, the second coupling mechanism configured to removably couple to a collection vessel receiving the expressed breast milk from the pumping device; and
a sensor coupled to the housing, the sensor configured to measure one or more aspects of the expressed breast milk as the expressed breast milk passes from the pumping device through the central channel into the collection vessel.

2. The apparatus of claim 1, further comprising a processing unit in communication with the sensor.

3. The apparatus of claim 2, wherein the processing unit comprises a communication module configured to communicate with one or more of a computing device or a server.

4. The apparatus of claim 3, wherein the communication module is configured to transmit measurement data generated by the sensor to one or more of a computing device or a server.

5. The apparatus of claim 3, wherein the processing unit is configured to analyze measurement data generated by the sensor, and wherein the communication module is configured to transmit the analyzed measurement data to one or more of a computing device or a server.

6. The apparatus of claim 1, further comprising a power source operatively coupled to the sensor.

7. The apparatus of claim 1, wherein the housing comprises an exterior portion and an interior portion, the exterior portion comprising the first coupling mechanism and the interior portion comprising the sensor and the second coupling mechanism, and wherein the exterior portion and the interior portion are coupled together.

8. The apparatus of claim 7, wherein the exterior portion and the interior portion are fixed in position relative to one another during operation of the apparatus.

9. The apparatus of claim 1, wherein the housing comprises a shroud surrounding one or more of the sensor, a processing unit, and a power source, thereby protecting one or more of the sensor, processing unit, and power source from external forces.

10. The apparatus of claim 1, wherein the first coupling mechanism or the second coupling mechanism comprises screw threads, quarter turn couplings, bayonet couplings, or interference fits.

11. The apparatus of claim 10, wherein the first coupling mechanism comprises male screw threads, and the second coupling mechanism comprises female screw threads.

12. The apparatus of claim 1, wherein the sensor comprises a strain gauge.

13. The apparatus of claim 12, wherein the strain gauge is disposed on a lateral member of the housing, the lateral member coupled to the second coupling mechanism, and wherein the strain gauge is configured to measure strain placed on the second coupling mechanism by the collection vessel coupled thereto, thereby generating measurement data indicative of a volume of expressed milk.

14. The apparatus of claim 13, wherein the housing is configured to isolate a load exerted by the collection vessel.

15. The apparatus of claim 14, wherein the housing comprises a lip coupled to the lateral member, the lip configured to engage a top surface of the collection vessel coupled to the second coupling mechanism, such that the top surface of the collection vessel is flush against a bottom surface of the lip.

16. The apparatus of claim 13, wherein the lateral member is disposed annularly around the central channel.

17. The apparatus of claim 13, wherein the lateral member comprises one or more elongated beams.

18. The apparatus of claim 13, wherein the strain gauge comprises a plurality of strain gauges disposed on a plurality of locations on the lateral member.

19. The apparatus of claim 12, further comprising a valve coupled to the housing and configured to permit passage of the expressed breast milk through the central channel, wherein the strain gauge is disposed on the valve, and wherein the strain gauge is configured to measure strain placed on the valve by the expressed breast milk, thereby generating measurement data indicative of a volume of expressed milk.

20. The apparatus of claim 1, wherein the sensor comprises an accelerometer.

21. The apparatus of claim 20, further comprising a valve coupled to the housing and configured to permit passage of the expressed breast milk through the central channel, wherein the accelerometer comprises a valve accelerometer disposed on the valve, and wherein the valve accelerometer is configured to measure a motion of the valve, thereby generating measurement data indicative of a volume of expressed milk.

22. The apparatus of claim 21, further comprising a background motion accelerometer coupled to a portion of the housing, the background motion accelerometer configured to measure a motion of the apparatus.

23. The apparatus of claim 22, wherein the motion of the apparatus is subtracted from the motion of the valve to determine a normalized motion of the valve.

24. The apparatus of claim 1, wherein the sensor comprises a beam-break sensor configured to detect passage of the expressed breast milk through the central channel.

25. The apparatus of claim 24, wherein measurements generated by the beam-break sensor are used to determine a volume of the expressed breast milk.

26. The apparatus of claim 25, wherein measurements generated by the beam-break sensor are used to determine a composition of the expressed breast milk.

27. The apparatus of claim 1, wherein the sensor comprises an image sensor configured to detect passage of the expressed breast milk through the central channel.

28. The apparatus of claim 27, wherein the image sensor comprises a charge-coupled device (CCD) configured to count drops of the expressed breast milk passing through the central channel, thereby generating measurement data indicative of a volume of expressed milk.

29. The apparatus of claim 1, wherein the sensor comprises a capacitive sensor configured to detect passage of the expressed breast milk through the central channel, thereby generating measurement data indicative of a volume of expressed milk.

30. A system for measuring expressed breast milk, the system comprising:
a pumping device configured to express breast milk from one or more breasts;
a collection vessel configured to receive the expressed breast milk; and
a sensing adaptor comprising a first end and a second end opposite the first end, the first end removably coupled to the pumping device and the second end removably coupled to the collection vessel, the sensing adaptor comprising a sensor configured to measure one or more aspects of the expressed breast milk as the expressed breast milk passes from the pumping device through a central channel of the sensing adaptor into the collection vessel.

31. The system of claim 30, further comprising a computing device in communication with the sensing adaptor, the computing device configured to receive measurement data generated by the sensor.

32. The system of claim 31, wherein the computing device comprises a smartphone, a tablet, a desktop computer, or a laptop computer.

33. The system of claim 31, wherein the computing device comprises a processing unit having instructions stored thereon for performing an analysis of the measurement data.

34. The system of claim 31, wherein the computing device comprises a user interface configured to display the measurement data to a user.

35. The system of claim 30, further comprising a remote server in communication with the sensing reservoir or the computing device, the remote server configured to perform one or more of analysis of the measurement data and storage of the measurement data.

36. A method for measuring expressed breast milk, the method comprising:
providing a pumping device, a collection vessel, and a sensing adaptor having a first end and a second end opposite the first end, the first end comprising a coupling mechanism configured to couple to the pumping device, and the second end comprising a second coupling mechanism configured to couple to the collection vessel;
coupling the first coupling mechanism of the sensing adaptor to the pumping device;
coupling the second coupling mechanism of the sensing adaptor to the collection vessel;
expressing breast milk from the breast using the pumping device;
generating, via a sensor coupled to the sensing adaptor, measurement data indicative of one or more aspects of the expressed breast milk.

37. The method of claim 36, wherein the sensing adaptor further comprises a processing unit, and the method further comprises analyzing measurement data generated by the sensor with the processing unit.

38. The method of claim 36, wherein the sensing adaptor further comprises a communication module, and the method further comprising transmitting measurement data generated by the sensor to one or more of a computing device or a server, via the communication module.
